(19) United States
(12) Patent Application Publication
(10) Pub. No.: US 2012/0095355 A1
(43) Pub. Date: Apr. 19, 2012

(54) Volume Sensing

(75) Inventor: Mark Zdeblick, Portola Valley, CA (US)

(73) Assignee: PROTEUS BIOMEDICAL, INC., Redwood City, CA (US)

(21) Appl. No.: 12/678,147

(22) PCT Filed: Mar. 13, 2010
(86) PCT No.: PCT/US2010/027261

§ 371 Date: Jul. 19, 2011

Related U.S. Application Data
(60) Provisional application No. 61/160,265, filed on Mar. 13, 2009.

(51) Int. Cl. A61B 5/053

(52) U.S. Cl. 600/508; 600/547

(57) ABSTRACT

Each sensor in one or more pairs of sensors is associated with a particular tissue, for example a first tissue location and a second tissue location respectively. Tissue at a particular tissue location may be solid, for example, muscle tissue, fat tissue, etc., or fluid, for example, blood, fluids associated with edema, etc. The area between the two tissue locations associated with a pair of sensors may comprise solid tissue, fluid tissue, an empty chamber, or combinations thereof. For each pair of sensors in the at least one pair of sensors, a first impedance measurement between the pair of sensors and associated with a first frequency is determined. For each pair of sensors in the at least one pair of sensors, a second impedance measurement between the pair of sensors and associated with a second frequency is determined. A comparison of a ratio of the first impedance measurement at a point in time to the second impedance measurement at a corresponding point in time may be made to determine a volume-related value associated with an area located between the first tissue location and the second tissue location.

This application claims the benefit of U.S. patent application No. 61/235,979 filed Mar. 13, 2009, which application is incorporated herein by reference for all purposes.

### BACKGROUND

It would be very helpful if organ volumes could be measured nearly continuously and in real time and in spite of bodily movements and variations in body position. This is not, however, easy to do. Until now, it has only been possible to measure organ volumes in very constrained settings and in very constrained body positions.

Cardiac resynchronization therapy (CRT) and various other cardiac therapies may optimize or improve cardiac performance. The cardiac performance may be gauged, for example, by assessment of various cardiac parameters such as tissue motion and blood volume.

In current practice, some cardiac properties may be approximated via external measurements. In one example, external ultrasound measurements are used to calculate some tissue parameters. Current use of ultrasound techniques, however, has been limited to wall position determination via external ultrasonography.

A potential drawback to the use of current ultrasonic techniques is that the techniques are typically restricted to ad hoc procedures, performed in a clinical setting. Thus, a patient's cardiac parameters are available, if at all, only during a specific time interval and are not available on an ongoing, for example continuous, basis.

Yet another potential drawback is that the patient typically undergoes the ultrasonic procedure immobilized in a supine position. Thus, the patient's cardiac activity reflects the position-relative parameter only. It will thus be appreciated that this makes external ultrasonography an inadequate tool for measuring cardiac parameters over a range of types of activities and postures.

It would be advantageous to have means to accurately ascertain various cardiac-related parameters associated with the patient in real-life situations and to receive the cardiac-related parameters on a real-time and/or continuous basis. If only such a means could be devised, this would represent an important advance in medical therapies to derive such clinical information, finding application in various technology areas, including managed cardiac care.

### SUMMARY OF THE INVENTION

Each sensor in one or more pairs of sensors is associated with a particular tissue, for example a first tissue location and a second tissue location respectively. Tissue at a particular tissue location may be solid, for example, muscle tissue, fat tissue, etc., or fluid, for example, blood, fluids associated with edema, etc. The area between the two tissue locations associated with a pair of sensors may comprise solid tissue, fluid tissue, an empty chamber, or combinations thereof. For each pair of sensors in the at least one pair of sensors, a first impedance measurement between the pair of sensors and associated with a first frequency is determined. For each pair of sensors in the at least one pair of sensors, a second impedance measurement between the pair of sensors and associated with a second frequency is determined. A comparison of a ratio of the first impedance measurement at a point in time to the second impedance measurement at a corresponding point in time may be made to determine a volume-related value associated with an area located between the first tissue location and the second tissue location.

### DESCRIPTION OF THE DRAWING

FIG. 1 shows a sensing arrangement in an organ of a subject.

FIG. 2 shows a lead which might be used for such sensing.

FIG. 3 shows a detail of such a lead.

### DETAILED DESCRIPTION

What will be described is a device, system, and method for determining various parameters, including tissue volume and fluid volume. In one aspect, for example, a multiplex lead such as a cardiac volume-sensing lead may estimate an amount of fluid, for example the amount of blood pumped out of heart chambers. The estimation may be adaptable for various uses, including CRT optimization and various other therapies.

Furthermore, methods of the invention may be carried out in any suitable body structure, such as but not limited to the heart, arterial or venous vasculature, and other body structures. Examples of body structures include tissue, such as cardiac tissue, and organs, such as the urinary bladder, stomach, lungs, etc.

Various applications will be readily apparent, such as, for example, measuring the congestion in the lungs, determining how much fluid is in the brain, assessing distention of the urinary bladder, assessing content volume of the stomach, assessing edema or blood pooling associated with a limb, etc.

Various aspects may utilize electrical plethysmography technology. Generally, electrical plethysmography technology detects electrical energy generated by fluid, for example, fluid absence, fluid presence, and/or fluid flow. More particularly, electrical field(s) may be generated. Impedance measurement(s) at different frequencies may be determined between various points in an electrical field. The impedance measurement(s), alone or in combination with other data, may be used to derive various information and to inform various decisions.

In one scenario an electrical field is generated and applied externally, internally, or a combination of both to at least a portion of a living being.

With that scenario, various applications and determinations may be made via various methods, using various devices, systems, and combinations of the foregoing.

In one example, each sensor in one or more pairs of sensors is associated with a particular tissue, for example, a first tissue location and a second tissue location respectively. Tissue at a particular tissue location may be solid, for example, muscle tissue, fat tissue, etc., or fluid, for example, blood, fluids associated with edema, etc. The area between the two tissue locations associated with a pair of sensors may comprise solid tissue, fluid tissue, an empty chamber, or combinations thereof.

For each pair of sensors in the at least one pair of sensors, a first impedance measurement between the pair of sensors and associated with a first frequency is determined. A sequence of such impedance measurements can be determined over a first time interval, for example t1, t2, t3, etc.

For each pair of sensors in the at least one pair of sensors, a second impedance measurement between the pair of sensors and associated with a second frequency is determined. A sequence of such impedance measurements can be determined over a second time interval, for example, t4, t5, t6, etc.

A comparison of a ratio of the first impedance measurement at a point in time to the second impedance measurement at a corresponding point in time may be made to determine a volume-related value associated with an area located between the first tissue location and the second tissue location. For example, the first impedance measurement at t1 is compared with the second impedance measurement at t4; the first impedance measurement at t2 is compared with the second impedance measurement at t5; and the first impedance measurement at t3 is compared with the second impedance measurement at t6, etc.

The volume-related value (ratio) may be compared against known values or further manipulated to derive and inform various values and conclusions. For example, a pair of sensors is located variously respective to a wall of the right ventricle, where an impedance ratio of 90% may indicate a tissue volume associated with solid muscle tissue. If ratios at t1/t4, t2/t5, and t3/56 are 30%, 60%, and 90%, respectively, the 90% volume-related value, which equates to solid tissue may indicate virtually complete contraction of the right ventricle, resulting in a high ejection fraction of blood from the right ventricle during systole. Such a high ejection fraction (90%) may be interpreted as an indicator of efficient pumping action of the heart. Further, analysis and comparison of ratios over time interval and/or analysis and comparison of multiple ratios associated with multiple pairs of sensors may further enhance data, for example, volume-related data, frequency-related data, for example, at what rate the ejection fraction improves over time if a cardiac patient's treatment regimen is adjusted.

In one example, a lead device may be employed to accomplish the foregoing. The lead device may comprise, for example, at least one pair of sensors capable of association with a respective first tissue location and a respective second tissue location and a logic module to (for each pair of sensors in the at least one pair of sensors) determine at a first point in time a first impedance measurement associated with a first frequency between the at least one pair of sensors; determine at a second point in time a second impedance measurement associated with a second frequency between the at least one pair of sensors; and compare a ratio of the first impedance measurement to the second impedance measurement to determine a volume-related value associated with an area located between the first tissue location and the second tissue location.

Additionally, the logic module may determine, over a first time interval, a first sequence of impedance measurements associated with a first frequency; determine, over a second time interval, a second sequence of impedance measurements associated with a second frequency; and compare ratios of measurements of the first sequence to corresponding measurements of the second sequence to determine volume-related data and frequency data associated with an area located between the first tissue location and the second tissue location.

Such a device may further include a ratio comparison module and/or a field generation module. The ration comparison module may compare each ratio of measurements associated with each pair of sensors in the at least one pair of sensors to determine to determine volume-related data and frequency data associated with all the pairs of sensors in the at least one pair of sensors. The field module may generate an electrical field of alternating current. The electrical field of alternating current may be applied in at least one of the following manners: externally to the body structure; internally to the body structure; and externally and internally to the body structure.

A system may comprise, for example, a lead device and a communication module to convert the measurements to a destination device-compatible format and to communicate the converted measurements to a destination device.

Additionally, the system may comprise the destination device, for example, a can.

Further, the system may comprise a delivery element to generate an electrical field. The generated electrical field, impedance between two tissue locations may be measured over a time sequence to generate a series of measurements, for example, sampling at two different frequencies. The frequencies, for example, may be a relatively high frequency and a relatively low frequency. An example of a relatively high frequency is on the order of 100 kHz. An example of a relatively low frequency is on the order of 10-15 kHz. It will be recognized that other methods and frequencies may be employed.

To illustrate, a field of alternating current (AC) may be generated across a structure such as the heart. From measurements of the strength of the AC field at various points, electrical impedance between those points may be determined. At certain frequencies, the electrical conductivity of cardiac tissue differs from the electrical conductivity of blood tissue. By determining the impedance between various points as a function of time, a ratio of the amount of cardiac tissue to blood tissue between each of those points as a function of time may be ascertained. From a comparison of the ratios, estimations of blood volume in the heart chambers may be generated. Comparisons of the estimations over a temporal period may provide further information, for example, information from which diagnoses and/or clinical inferences may be drawn. One example of a calculation from which a clinical inference may be drawn is ejection fraction, i.e., an amount of blood determined to be ejected from a heart chamber. Ejection fraction is often used, for example, in clinical practice as an indicator of overall and/or specific cardiac performance.

More particularly and in various aspects, more than one frequency may be used to set up the AC field to take advantage of the difference in the variation of impedance as a function of frequency between fluid and solid substances at various frequencies. In various aspects, the AC field may be applied externally or internally to the body structure, each of which is described hereinafter.

Examples of such leads, components, etc., include, but are not limited to, those disclosed/described in United States patent publication numbers 2006-0058588 A1, 2006-0116581 A1, 2006-0217793 A1, 2007-0123944 A1, 2007-0135721 A1, 2007-0161894 A1, 2008-0183072 A1, 2008-0058656 A1, 2008-0255647 A1, 2008-0294218 A1, 2009-0299447 A1, 2008-0208068 A1, 2009-0036769 A1, the disclosures of which are incorporated herein by reference in their entirety and for all purposes.

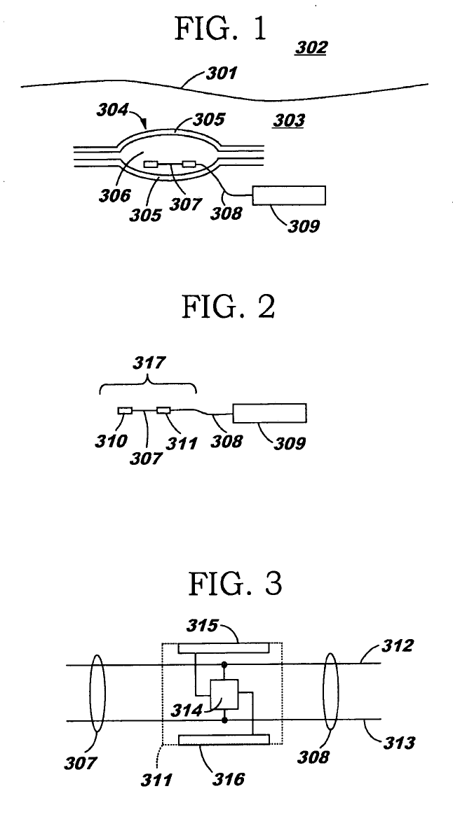

FIG. 1 depicts an embodiment of the invention as employed in an animal subject such as a human being. Epidermis 301 distinguishes air 302 from the body 303 of the subject. Within the body 303 is an organ 304 composed in part of tissue 305. The organ 304 might be a heart (as mentioned in examples below) or might be any of several other organs such as a bladder or stomach. In this example the heart 304 has an interior 306 containing blood (which in this context may be considered to be a distinct tissue).

As mentioned above there are a number of therapeutic or diagnostic goals which may be better served if a way may be found to measure the liquid volume within the organ 204, or to carry out any of a range of measurements providing a direct or indirect indication thereof. For example it may be very helpful to be able to arrive at some estimate of the ratio of first tissue (for example blood) to second tissue (for example heart muscle) in a particular region. Shifts in the ratio, upward or downward, may be indicative of changes in the liquid volume within the organ.

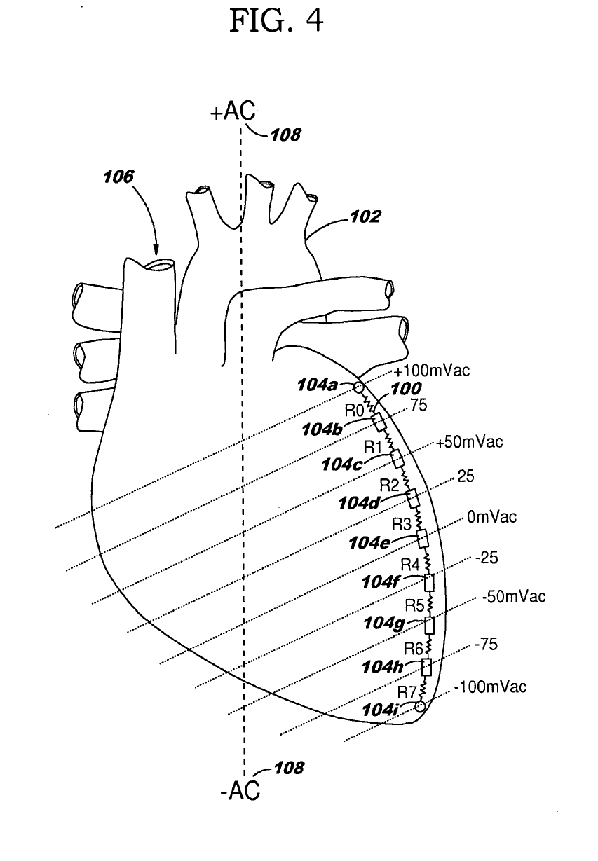

In this embodiment a lead 317 (FIG. 2) is provided having satellites 310, 311 about which more will be said below. The lead is shown in FIG. 1 with a first portion 308 and a second portion 307, the first portion connecting with a can 309 and with the second portion depicted within the organ 304.

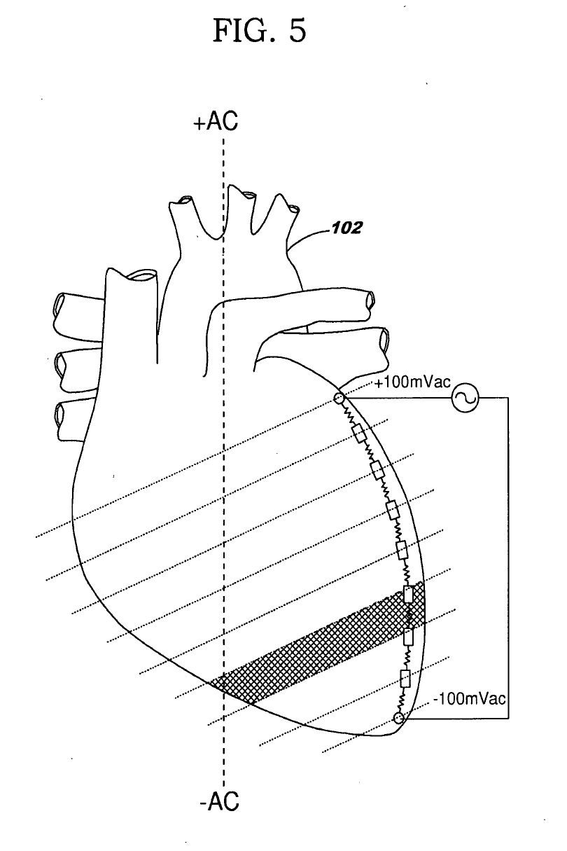

FIG. 3 gives an exemplary functional block diagram for satellite 311 and nearby structure. First portion 308 contains first and second conductors 312, 313 which connect with satellite 311 and which pass through to second portion 307. Within satellite 311, the conductors 312, 313 connect with integrated circuit chip 314. Chip 314 is connected with electrodes 315, 316 which are able to be in contact with tissues or liquids nearby.

In a preferred embodiment the chip 314 draws power from the conductors 312, 313, and receives commands by means of those conductors, for example from can 309. The commands are addressable, so that the can 309 can emit a command that is acted upon by a single satellite 310 or 311, and using a suitable protocol other particular commands are acted upon by more than one satellite. Particular commands may prompt a chip 314 to couple one of the electrodes 315, 316 to one of the conductors 312, 313. Other commands might prompt a chip 314 to respond by means of a message response back to the can 309.

It will be appreciated, upon consideration of the discussion herein, that the number of satellites need not be two as in FIGS. 1 and 2 but might be some other number. Likewise the number of electrodes at a particular satellite need not be two as in FIG. 3 but might be one or more than two. While it is thought to be preferable for the device at the end of the lead to be a can 309 implanted within the body, other arrangements are possible, for example if the lead passes through the boundary 301 and connects with electronic equipment that is external to the body 303. These and other variants would not depart in any way from the invention.

It will also be appreciated that the lead 317 could be a very simple lead lacking any electronics at all, and composed of metallic conductors and metallic electrodes.

An exemplary sequence of events will now be described. Electrical energy is passed from satellite 310 and to satellite 311. The current is measured, and the voltage drop across the satellites is measured. The ratio of voltage and current defines the measured impedance therebetween. In this way the impedance of tissues nearby to the satellites 310 and 311 is measured. As it turns out, distinct tissues such as blood or heart muscle often have distinct impedances that differ as a function of the frequency of the emitted energy. For example at a higher frequency or frequency band the impedances may be nearly the same for both types of tissue, and at a lower frequency or frequency band the impedances of the heart tissue and the blood may differ, for example the impedance of the heart tissue may be lower than the impedance of blood.

It will thus be appreciated that an impedance in one band (or at a first frequency) to impedance in a second band (or at a second frequency) may be an indicator of the blood volume within the organ. If at a particular moment there is less blood, then more of the tissue nearby to the lead will be heart muscle, giving rise to a lower impedance. If at a different particular moment there is more blood, then legs of the tissue nearby to the lead will be heart muscle, giving rise to a higher impedance.

The measured ratio may, with sufficient calibration and application of corrective factors, permit arriving at a credible measurement of the absolute volume of liquid within the organ. But the invention can offer many benefits even if one were not to set a goal of arriving at a measurement of absolute volume. For example merely tracking trends of the ratio over time may provide extremely helpful information for diagnostic or therapeutic purposes. To give a simple example a particular dosage of a drug may correlate with a particular measured ratio, with a first increase in the dosage giving rise to a corresponding change in the measured ratio. Yet a second further increase in the dosage might not give rise to any further change in the measured ratio. This might permit a decision not to continue administering the drug at the level of the second increase.

Similarly such tracking of trends in the ratio might permit screening candidate drugs so as to distinguish between drugs which (on the one hand) correlate with desired changes in the ratio, and which (on the other hand) do not correlate with changes in the ratio, or which are seen to correlate with changes in the ratio that are in the opposite direction to the desired direction.

Frequent measurements (for example several measurements per second) may permit arriving at some indication of the organ volume at high or low points, for example at points during a pumping cycle in a heart. This may in turn permit tracking heart function over time, so as to aid in prediction of failure or to help with other diagnostic or therapeutic goals.

In a relatively simple embodiment, the measurement process can start with measurement of impedance at a first frequency (or within a first frequency band), and can continue at a later time with measurement of impedance at a second frequency (or within a second frequency band). In such a simple embodiment, what might happen is that events such as beating of a heart or other physical movements could perturb one measurement at some frequency relative to an earlier or later measurement at some other frequency. Such perturbations could give rise to “noise” superposed over the desired “signal” (measured ratios).

The stimulation energy could be emitted more or less continuously or could be emitted in bursts or pulses or chirps, depending on factors such as the energy budget available and the extent to which values measured at intervals may serve the diagnostic or therapeutic needs without the need for continuously measured values.

FIGS. 4 and 5 provide views of a cardiac volume-sensing lead 100 associated with a heart 102. In this example there is an externally-applied AC field 108 as shown. The lead includes a number of electrodes 104, for example, electrodes 104a-104i, at various locations and a two-wire bus associated with a can (omitted for clarity in FIGS. 4 and 5). The two-wire bus may go down the lead and may connect to each of the electrodes.

In each electrode 104 for example, electrodes 104a-104i, there may be an AC voltage converter (not shown) measuring the AC voltage at that frequency and converting the voltage measurement to a digital number. Each electrode may transmit the digital number up the two-wire bus and back to the can.

Rather than providing an AC voltage converter at each electrode position, another option is to program the electrodes so that only one or two electrodes 104 are connected to the two-wire bus, and in this way the electric field at one or two electrodes may be transmitted up the bus to a central controller (not shown). Inside the central controller, electronics may filter the signal(s) into separate frequencies and determine the amplitude of each of the frequencies of each of the signals on each of the wires. The central controller may convert the amplitudes into digital values.

In various aspects, the AC field 108 may be applied externally to the heart, for example, across the top and bottom of the heart 102. The voltages may be monitored at the electrodes 104, for example, electrodes 104a-104i. A ratio of the voltages may be determined from the voltages monitored at each electrode 104, i.e., the voltages at the electrodes change based on the relative ratios of the impedance between them. For example, the AC field 108 is driven such that the distal electrode, for example, the electrode 104i, is at −100 mVAC and the proximal electrode, for example, the electrode 104a, is +100 mVAC. If, by way of example and not limitation, the eight electrodes 104 are relatively evenly spaced and the impedance between the electrodes is uniform, the middle electrode, for example, the electrode 104c, stays at 0 mV AC. Thus, electrodes 104 may be relatively evenly spaced at points between the proximal electrode and the distal electrode, for example, the electrode 104b is at 75 mV AC, the electrode 104c is at 50 mV AC, the electrode 104d is at 25 mV AC, the electrode 104e is at 0 mV AC, the electrode 104f is at −25 mV AC, the electrode 104g is at −50 mV AC, the electrode 104h is at −75 mV AC. (The negative and positive voltage assignments are used in a phase sense.)

The AC field 108 in the vicinity of the electrodes 104 may not vary uniformly due to the heterogeneous composition of the heart tissue. As a result, the impedances between electrodes 104 are neither uniform nor static. As such, the voltages sampled at each electrode 104 correspond to the relative impedance between each of the electrodes 104 associated with various locations of the heart 102.

To determine volume, the voltages may be measured at each of the electrodes 104. From each of the measured voltages, an effective resistance (here, an impedance) may be calculated and the resistance of the cardiac tissue caused by current flowing through the tissue from the broadcast electrodes but as sampled by these electrodes may be derived.

In various aspects, a voltage map showing each of these electrode points may show some curvature and some variation, and the voltages at these different electrode points may be sampled. The amount of voltage changing between such electrode points can be affected by the motion of the lead 100 through the heart 102, thus there may be some inaccuracies if the voltage field alone is used. However, the change between these electrode points may also be governed by a change in impedance of blood tissue and cardiac tissue. Generally, the impedance of blood tissue and cardiac tissue is very similar at relatively high frequencies and the impedance of blood tissue and cardiac tissue differs at relatively low frequencies.

Generally, when there is a significant amount of blood tissue in the chamber, for example, the left ventricle (LV), the voltage drop between the electrodes 104 is different than when there is a relatively insignificant amount of blood tissue in the chamber. During a systolic phase, for example, relatively little blood tissue is present in the LV. The septum and left wall of the heart contract, thus the LV is relatively small in size with a significant amount of cardiac tissue present in an area normally occupied by blood tissue during diastole. Thus, during the systolic phase, these lines tend to change in terms of where the voltages are that are actually dropped.

In various aspects, voltages may be measured at multiple frequencies. To illustrate, voltages are measured at 10 kHz as well as 100 kHz. In another illustration, voltages are measured at 500 kHz and one MHz. Multiple frequency measuring samplings and the voltages at each of those frequencies may be converted to a digital number at predetermined time intervals, for example, approximately 100 to 400 times per second. Each of the electrodes 104 may broadcast the respective digital number down the two-wire bus to the can.

In various aspects, various protocols/schemes may be used to convey the digital number information to the can. In one example, a time multiplexing scheme may be employed, wherein each electrode 104 has a point in time in which the electrode 104 may broadcast a signal. In another example, a frequency multiplexing scheme may be employed, wherein each electrode 104 can be given a different frequency on which to broadcast a signal having the digital number information to the can.

In various aspects, it may be beneficial to have multiple AC fields 108 applied at different directions to cancel out various effects, for example, effects of the lead moving back and forth, or to measure the impedances in different orientations.

FIG. 5 provides a view of the cardiac volume-sensing lead 100 of FIG. 1 associated with the heart 102 having an internally-applied field 108, according to an embodiment of the invention. The lead 100 includes a number of electrodes at various locations.

In various aspects, the AC field 108 may be applied internally. In this manner, the two wire bus may be used as both an effector of stimulation, for example, a voltage-delivering or current-delivering element, as well as a voltage-sampling element, as heretofore described.

To illustrate, an electrode at one end of may drive a certain amount of AC current relative to an electrode at the other end to develop a desired voltage, for example giving rise to a momentary +100 mV value at one end and a −100 mV value at the other end.

In one example, 100 mV may be used and, presumably, less current may be needed (as compared with an externally applied field) because the current need not go through the skin to set up a field. The various electrodes in between the two ends may be sampling the voltage locally and then communicating the information back to the bus and thence to analysis electronics.

In various aspects, the internally-applied field 108 may not have any external electrodes at all. The lead, for example, with its electrodes, can be completely implanted within the heart 102 and the volume change output can be monitored at any time. In this manner, real time and/or ongoing cardiac output data may be provided.

In various aspects, the lead 100 may be at least partially located in the cardiac vein over the LV and the electrode(s) 104 on the lead 100 may be used to stimulate the heart tissue, for example, for CRT benefit. In this manner, the lead 100 may be used for measuring heart health and may provide information used to for various purposes. Purposes include estimating when to replace a valve, providing feedback used to prescribe medications, providing feedback used to determine efficacy of medications, etc.

In various aspects, the lead 100 may be used for measuring regurgitant flows, for example, depending on the extent of coordination with the backward flow of blood tissue back into the lung area. Thus, the ratio of impedance of each of these particular electrode points may be measured as such measurements correspond to the ratio of blood tissue and volume.

To illustrate, assume that blood tissue 306 and cardiac tissue 305 have significantly different impedances. During a diastolic phase the heart relaxes, expanding the chambers and permitting entry of a significant volume of the blood tissue 306 into the LV chamber. Assume there is a significant volume of the blood tissue 306 between two measurement points and a relatively insignificant volume of cardiac tissue 305. Assuming the blood tissue 306 is more resistive at this particular frequency than the cardiac tissue 305, then a significant amount of voltage will develop between these two points for the given current, that is, indicating a higher overall impedance. The amount of voltage for a given current will be relatively high, which may be used as an indicator of various states and/or parameters, for example, diastole, volume of blood at a point in time, volume of blood over a time interval, etc.

During a systolic phase the heart 102 contracts, thickening the heart wall, for example, the cardiac tissue 305, and ejecting blood tissue, for example, the blood tissue 306 from the chambers. The blood tissue 306 is assumed to be more resistive at this particular frequency than the cardiac tissue 305. If there is an insignificant volume of the blood tissue 306 and a relatively significant amount of the cardiac tissue 305, a lower impedance value is derived because relatively less voltage drop develops across the two electrodes for a given amount of current going between the two electrodes and the associated tissue. Thus, from any two electrodes 104, an estimation of the ratio of blood tissue and cardiac tissue may be derived. From the ratio, an estimation of the amount, for example, volume, of blood tissue present in a chamber, for example, the LV chamber, may be made.

Various aspects include a multiplex system that can be implemented as a pacing device, as an implanted device, or as a combination thereof.

Various aspects provide both the current as well as sampling the voltages at each of those locations. Additional features include electrodes that have the electronics necessary to convert the signals from an AC voltage into a digital number that can be transmitted up the bus 106, for example, the two-wire bus, and into the can.

In various aspects, feedback may be provided to a CRT optimization system which may assist in determination(s) as to preferred cardiac placement locations to provide stimulation and the time thereof.

In various aspects, the lead 100 may be associated with one or more heart chambers, for example, located in the cardiac vein over the left ventricle, located on the right side of the heart, etc. Because the ratio of blood tissue in the heart to the cardiac tissue is being measured and the signals are not the electrical currents between any of the pairs of electrodes 104, application is not confined to any ventricle or atrium but may be relative to the entire heart. Thus, to a greater or lesser degree, sampling of multiple voltages at multiple locations and times may be occurring, depending on where the electrodes are actually placed.

To illustrate, a lead (not shown) may be placed at least partially into the right ventricle (in addition to the lead 104 that goes to the left ventricle). The lead placed in the right ventricle may have an array of electrodes associated with the right ventricle as well. The array of electrodes may further sample the fields at respective locations either in conjunction with the lead 104 in the left ventricle or separately to have a separate measurement(s) of the cardiac blood volume in the heart made only with the lead placed in the right ventricle. Moreover, measuring cardiac volumes may be accomplished using a lead in the cardiac veins as opposed to a catheter in the ventricle.

In various aspects, the lead may be configured with various numbers of electrodes. Generally, higher numbers of electrodes may provide greater resolution in terms of measuring the ratio of blood and volume between any two electrodes. Geographic separation of the electrodes may provide coverage of a greater area of the heart and thus may provide estimates of the amount of blood between the points of this greater area of the heart.

In various aspects, the number of frequencies may also vary. There may be user-determinable and configurable decision points with respect to numbers of electrodes, electrode locations, frequencies, etc. For example, there may be diminishing returns for more electrode locations, beyond a certain amount of locations, as opposed to fewer. There may be diminishing returns for greater numbers of frequencies, beyond a certain amount of frequencies used, as opposed to fewer. For example, various leads may be configured with eight electrodes, sixteen electrodes, four electrodes, five electrodes, six electrodes, etc., depending on the desired results.

Illustrative applications of the present invention include the following examples. To calibrate, two electrodes or satellites may be positioned relative to the cardiac wall or cardiac vein and near one another. The satellites sample tissue and get a ratio of the frequencies associated with the tissue locations. The right atrium is sampled, which may include predominantly blood, to determine a ratio. These measurements may be used as benchmarks. One is predominantly blood tissue and one is predominantly solid tissue. Any unknown value will give ratio of percentage of blood to tissue. For example, in the right ventricle there may be an electrode attached to the wall and two electrodes, for example, satellites, on the lead itself sampling the blood surrounding the satellites. For the solid tissue we put two electrodes/satellites in the cardiac vein for measure of the amount of impedance between those two neighboring satellites. The amount of solid tissue/blood tissue ratio across the ventricle is determined, where there may be, for example, an electrode in the right ventricle, in the right ventricular septum and at least one electrode/satellite in the cardiac vein over the left ventricle. The impedance ratio between those two gives us the amount of blood tissue relative to tissue between those two points. Because this changes over time, it is related to cardiac volume, for example, to measure a parameter such as ejection fraction. With an asynchronous cardiac condition, i.e., less than optimal time of contraction of a heart wall versus time of contraction of the septum, the same modeling process may be used. The modeling may show a timing difference, for example, septum contracts first and then walls contract and septum is relaxing while walls contract, indicating less than optimal pumping action.

The information may further show the rate at which the numbers change, indicating the relative frequency at which a patient's health improves or degrades. In some patients, the heart is so far distended, a ratio of 30% or 40%, may be observed. Increasing the ratio to 50% in such a case is a marked improvement in health for such a patient. Thus, if such an increase can be occur over a relatively short period, a treatment regimen driving such improvement may be considered highly effective. Conversely, an relatively slow increase over time from an 85% rating to a 90% rating may indicate an opportunity for adjusting a treatment therapy to improve the rate at which the increase is seen.

Another feature may be the ability to generate, using the above-described device, system, and/or method, a relative metric of the entire heart across chambers, for example, right or left ventricle, right or left atrium, because multiple pairs of electrodes/satellites may be used

Another feature may be accurate measurements. It turns out that the impedance between the electrode points is very important, but the biggest voltage drop between any two prior art devices will occur right at the electrode interface. One way to eliminate or reduce inaccuracies associated with the voltage drop is to use two adjacent electrodes to sample. One of those electrodes is used either to source or sink current and the adjacent one samples the potential in the nearby tissue. This may be done in pairs. Thus, if there are two satellites on the same lead one sources or sinks current and they both may measure potential relative to S1 (one of two conductors in a two-wire bus). S1 will be a local ground for both of the electrodes and S2 (the other of two conductors in the two-wire bus) will provide the energy that is then converted into a current for sourcing or sinking.

Another feature includes health and cardiac tissue itself. If the sensor pair is located near an aneurism, the ratio itself will be low, and there will be very little blood. Thus, its change during the cardiac cycle will change very little, as well. Thus, such a pattern of ratios may indicate ischemic conditions.

Another feature involves “mapping” a given tissue region using multiple pairs of sensors, for example, electrodes and/or satellites, as heretofore described. To illustrate, multiple leads may be placed in/around the heart to to “map” blood going in and out of various regions of the heart.

Other exemplary applications include bladder monitoring, for example, determining relative fullness of the bladder to inform further decisions or actions, for example, generate an email for patient to void, activate a device to stimulate voidance, etc. Similarly, stomach applications could permit informed actions and decisions based on the content volume of the stomach, for example, activate a constriction device, generate an email advising the patient to stop eating, etc.

Another feature is determination of overall volume of blood in the body by, for example, monitoring from one foot to another foot. Further inferences could include determinations of how much blood pooling or edema, for example, indicators of wellness related to diabetes, heart disease, etc. In one illustration, determination of excessive blood pooling in the leg may be followed by an email alert to the patient to get up and walk as a means to alleviate some of the pooling.

Those skilled in the art will have no difficulty devising myriad obvious improvements and variants of the invention, without departing from it. Such improvements and variants are intended to be encompassed within the claims which follow.

1. A method for use with a lead having at least first and second electrodes, and for use with circuitry having means for measuring impedance between the at least first and second electrodes at least two distinct frequency bands, the lead initially being sterile and contained within a sterile wrapper, the method comprising the steps of:
removing the lead from the sterile wrapper;
implanting the lead within an organ; and
connecting the lead to the circuitry.

2. The method of claim 1 further comprising the steps, performed after the connecting step, of measuring impedance at the at least two distinct frequency bands between the at least first and second electrodes, thereby arriving at an indication of the liquid volume of the organ.

3. The method of claim 1 wherein the impedance measurements at the at least two distinct frequencies happens at respective and distinct times.

4. A method for use with a lead having at least first and second electrodes, the lead disposed within an organ, the lead connected to first circuitry lacking means for measuring impedance between the at least first and second electrodes at least two distinct frequency bands, the method comprising the steps of:
disconnecting the lead from the first circuitry, and
connecting the lead to second circuitry, the second circuitry having means for measuring impedance between the at least first and second electrodes

5. The method of claim 4 further comprising the step, performed after the connecting step, of measuring impedance at the at least two distinct frequency bands between the at least first and second electrodes, thereby arriving at an indication of the liquid volume of the organ.

6. The method of claim 4 wherein the emission of the energy at the at least two distinct frequencies happens at respective and distinct times.

7. A method for use with a lead having at least first and second electrodes, the lead implanted within an organ, and for use with circuitry having means for measuring impedance between the at least first and second electrodes, the method comprising the steps of:
measuring impedance at the at least two distinct frequency bands at the first and second electrodes, and
applying a function to the sensed impedances, thereby arriving at an indication of the liquid volume of the organ.

8. The method of claim 7 wherein the emission of the energy at the at least two distinct frequencies happens at respective and distinct times.

9. A system comprising:
a lead having at least first and second electrodes;
circuitry having means for measuring impedance between the at least first and second electrodes;
the lead being sterile and contained within a sterile wrapper.

10. The system of claim 9, the circuitry characterized in that the emission of the energy at the at least two distinct frequencies happens at respective and distinct times.

11. A system comprising:
a lead having at least first and second electrodes;
circuitry having means for measuring impedance between the at least first and second electrodes;
the lead implanted within an organ of a living subject and connected with the circuitry.

12. The system of claim 11, the circuitry characterized in that the emission of the energy at the at least two distinct frequencies happens at respective and distinct times.

13. The system of claim 11 wherein the circuitry is implanted within the subject.

14. A method comprising:
associating each sensor in at least one pair of sensors with a respective first tissue location and a respective second tissue location; and
for each pair of sensors in the at least one pair of sensors:
determining at a first point in time a first impedance measurement associated with a first frequency between the at least one pair of sensors;
determining at a second point in time a second impedance measurement associated with a second frequency between the at least one pair of sensors; and
comparing a ratio of the first impedance measurement to the second impedance measurement to determine a volume-related value associated with an area located between the first tissue location and the second tissue location.

15. The method of claim 14, wherein the area comprises at least one of a solid tissue and a fluid tissue.

16. The method of claim 15, wherein the fluid tissue comprises at least one of a blood tissue and a non-blood tissue.

17. The method of claim 14, wherein the area forms an empty chamber between the first tissue location and the second tissue location.

18. The method of claim 14, further comprising:
determining, over a first time interval, a first sequence of impedance measurements associated with a first frequency;
determining, over a second time interval, a second sequence of impedance measurements associated with a second frequency; and
comparing ratios of measurements of the first sequence to corresponding measurements of the second sequence to determine volume-related data and frequency data associated with an area located between the first tissue location and the second tissue location.

19. The method of claim 18, further comprising:
comparing each ratio of measurements associated with a each pair of sensors in the at least one pair of sensors to determine to determine volume-related data and frequency data associated with all the pairs of sensors in the at least one pair of sensors.

20. The method of claim 14, further comprising:
generating an electrical field of alternating current.

21. The method of claim 14, wherein the generating an electrical field associated with a body structure comprises at least one of:
applying the electrical field externally to the body structure; and
applying the electrical field internally to the body structure.

22. The method of claim 14, wherein at least one of the first tissue location and the second tissue location is selected from a group consisting essentially of a heart, a bladder, a stomach, a brain, and at least one limb.

23. The method of claim 14, further comprising:
converting the measurements to a destination device-compatible format; and
conveying the converted measurements to a destination device.

24. The method of claim 10 wherein the communicating the converted measurements to a destination device comprises employing at least one of a time multiplexing scheme and a frequency multiplexing scheme.

25. A lead device, comprising:
at least one pair of sensors capable of association with a respective first tissue location and a respective second tissue location; and
a logic module to:
for each pair of sensors in the at least one pair of sensors:
determine at a first point in time a first impedance measurement associated with a first frequency between the at least one pair of sensors;
determine at a second point in time a second impedance measurement associated with a second frequency between the at least one pair of sensors; and
compare a ratio of the first impedance measurement to the second impedance measurement to determine a volume-related value associated with an area located between the first tissue location and the second tissue location.

26. The device of claim 25, wherein the area comprises at least one of a solid tissue and a fluid tissue.

27. The device of claim 26, wherein the fluid tissue comprises at least one of a blood tissue and a non-blood tissue.

28. The device of claim 25, wherein the area forms an empty chamber between the first tissue location and the second tissue location.

29. The device of claim 25, wherein the logic module is characterized in that it will:
determine, over a first time interval, a first sequence of impedance measurements associated with a first frequency;
determine, over a second time interval, a second sequence of impedance measurements associated with a second frequency; and
compare ratios of measurements of the first sequence to corresponding measurements of the second sequence to determine volume-related data and frequency data associated with an area located between the first tissue location and the second tissue location.

30. The device of claim 29, further comprises:
a ratio comparison module to compare each ratio of measurements associated with a each pair of sensors in the at least one pair of sensors to determine to determine volume-related data and frequency data associated with all the pairs of sensors in the at least one pair of sensors.

31. The device of claim 25, further comprising:
a field module to generate an electrical field of alternating current.

32. The device of claim 31, wherein the field module applies the electrical field of alternating current in at least one of the following manners:
externally to the body structure;
internally to the body structure; and
externally and internally to the body structure.

33. The device of claim 14, wherein at least one of the first tissue location and the second tissue location is selected from a group consisting essentially of a heart, a bladder, a stomach, a brain, and at least one limb.

34. A system comprising:
a lead device, having at least one pair of sensors capable of association with a respective first tissue location and a respective second tissue location;
a logic module to:
for each pair of sensors in the at least one pair of sensors:
determine at a first point in time a first impedance measurement associated with a first frequency between the at least one pair of sensors;
determine at a second point in time a second impedance measurement associated with a second frequency between the at least one pair of sensors; and
compare a ratio of the first impedance measurement to the second impedance measurement to determine a volume-related value associated with an area located between the first tissue location and the second tissue location; and
a communication module to convert the measurements to a destination device-compatible format and to communicate the converted measurements to a destination device.

35. The system of claim 34, further comprising the destination device.

36. The system of claim 35, wherein the destination device further comprises a can.

37. The system of claim 34, further comprising a delivery element to generate an electrical field.
